(19) United States
(12) Patent Application Publication
(10) Pub. No.: US 2013/0197327 A1
(43) Pub. Date: Aug. 1, 2013

(54) ANALYTE SENSOR

(71) Applicants: Rongsheng Chen, Oxford (GB); Andrew Farmery, Oxford (GB); Clive Hahn, Oxford (GB); Andrew Obeid, Oxford (GB)

(72) Inventors: Rongsheng Chen, Oxford (GB); Andrew Farmery, Oxford (GB); Clive Hahn, Oxford (GB); Andrew Obeid, Oxford (GB)

(73) Assignee: ISIS INNOVATION LIMITED, Summertown, Oxford (GB)

(21) Appl. No.: 13/806,090

(22) PCT Filed: Jun. 23, 2011
(86) PCT No.: PCT/GB2011/000945

§ 371 Date: Mar. 26, 2013

(30) Foreign Application Priority Data
Jun. 25, 2010 (GB) 1010768.8

(51) Int. Cl. A61B 5/1455

(52) U.S. Cl. CPC A61B 5/14556; A61B 5/14552
USPC 600/317

(57) ABSTRACT

An analyte sensor is disclosed that comprises:

a light source arranged for emitting light to a probe;

a detector arranged to receive fluorescence light emitted from said probe in response to the light incident from the light source, and to generate an output signal; and

a signal processor arranged to determine information related to the presence of an analyte at the probe based on at least the output signal of the detector,

wherein the probe comprises nano-particles comprising fluorescent material for which the fluorescence changes in response to the presence of analyte.

### RELATED FILINGS

This application is a U.S. national phase filing of PCT International Patent Application Serial No. PCT/GB2011/000945, filed Jun. 23, 2011, that is an international application of GB Application Serial No. 1010768.8, filed Jun. 25, 2010, the disclosures of which are incorporated herein by reference in their entirety.

### FIELD OF THE INVENTION

The present invention relates to a sensor for measuring an analyte, for example oxygen.

### BACKGROUND TO THE INVENTION

Sensors are known that use the principle of fluorescence quenching by an analyte. Excitation light is used to excite a dye which then emits fluorescence light. The presence of an analyte, such as oxygen, affects the fluorescence properties, typically quenching the fluorescence such that fluorescence lifetime and therefore the fluorescence intensity are reduced. By measuring the fluorescence response, the presence of the analyte can be detected and quantified.

However, the response time of conventional sensors to changes in analyte tends to be long, such as of the order of several seconds. This makes them unsuitable for measuring more rapidly changing phenomena, such as breath-by-breath analysis of oxygen levels. One attempt to overcome this problem is to make the fluorescent dye into an even thinner layer so that the diffusion time of the analyte in the sensing region is reduced, and so the response time of the sensor is also decreased. However, this has the problem that much of the excitation light passes straight through the fluorescent material, and also the intensity of the fluorescent light is reduced. This presents considerable practical difficulties in detecting the fluorescent light, and so can make the sensor less accurate or even unusable.

The present invention seeks to alleviate some or any of these problems.

### SUMMARY OF THE INVENTION

Accordingly, the present invention provides an analyte sensor comprising:
a light source;
a probe arranged to receive light emitted from the light source;
a detector arranged to receive fluorescence light emitted from said probe in response to the light incident from the light source, and to generate an output signal; and
a signal processor arranged to determine information related to the presence of an analyte at the probe based on at least the output signal of the detector,
wherein the probe comprises nano-particles comprising fluorescent material for which the fluorescence changes in response to the presence of analyte.

The response time of this sensor is rapid for a variety of reasons including the fact that the nano-particles present a very large surface area to volume ratio, which enhances the analyte diffusion speed.

The use of nano-particles also means that significant scattering of the excitation light occurs within the sensing region of the probe, which increases the quantum efficiency of the fluorescent material, and so improves the sensitivity and accuracy of the sensor.

Preferably the nano-particles are embedded in a matrix, such as silicone or PMMA, in which case the response time is rapid because the matrix has good permeability to the analyte because of its low solubility to the analyte and high diffusion rate. Preferably, the response time of the sensor is less than 300 ms, or even less than 200 ms.

Preferably the analyte sensor is an oxygen sensor.

### BRIEF DESCRIPTION OF THE FIGURES

FIG. 1 depicts schematically an analyte sensor of the invention;

FIG. 2 is an illustration of the probe tip of the analyte sensor of FIG. 1; and

FIG. 3 shows graphs of experimental results of the dynamic response of the sensor to oxygen.

### DETAILED DESCRIPTION OF THE INVENTION

The present invention provides an analyte sensor that uses fluorescence quenching measurements. A preferred embodiment relates to the measurement of oxygen concentration, as will be described in more detail below. This is merely an example, other analytes could be sensed, for example CO₂, N₂O, NO₂ and other Oxides of nitrogen (NOx).

Firstly, the general arrangement and operation of the sensor will be explained.

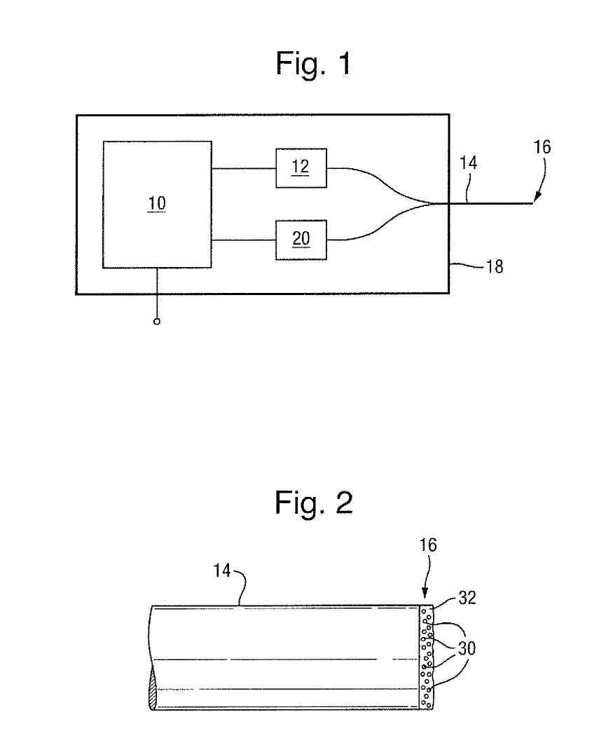

FIG. 1 shows schematically an embodiment of a sensor according to the invention. A controller 10 drives a light source 12 which generates the excitation light to be used for stimulation of fluorescent material being used to sense the analyte. The light source 12 can be, for example, an LED or laser diode. The output wavelength of the light source is chosen to suit the fluorescent material, described below, such that a transition in the material is stimulated; in the preferred embodiment the wavelength is from 450 nm to 503 nm. The term “light” is not intended to imply any particular restriction on the emission wavelength of the light source 12, and in particular is not limited to visible light. The light source 12 can include an optical filter to select a particular wavelength of excitation, but this filtering may be unnecessary if the light source has a sufficiently narrow band or is monochromatic.

The sensor also comprises a probe, which in this embodiment comprises an optical fiber 14, and the fluorescent material is located at the tip 16 of the probe. In preferred embodiments, the optical fiber 14 is made of glass (silica) or plastic, and typically has a diameter in the range of from 0.125 mm to 0.5 mm.

The light output from the light source 12 is transmitted to the probe tip 16 along the optical fiber 14. Appropriate couplers (not shown) are used to couple the light into and out of the optical fiber 14. The probe may also be removably connectable with the other components of the device that are provided in a housing 18.

The probe tip 16 is located in use in the environment in which the analyte is to be measured. The environment is not restricted to any particular phase, and could be, for example, gaseous (such as measuring oxygen levels in breath) or liquid (such as measuring dissolved oxygen in blood). In a preferred embodiment, the optical fiber 14 is made of a polymer, such as polymethylmethacrylate (PMMA) which is biocompatible and so can be inserted into a patient for measuring oxygen levels in tissues or body fluids. However, it is not essential to use an optical fiber; the transmission of light to and from the fluorescent material can be made by alternative means such as other forms of waveguide or free-space optics.

The fluorescent material at the probe tip 16 absorbs some of the excitation light received from the light source 12 and very shortly afterwards emits fluorescence light, typically at a longer wavelength. If the light source 12 were to emit a single pulse, then the intensity of the emitted fluorescent light would exhibit an exponential decay, and the half-life of this decay (the life time) is dependent on the ambient analyte concentration. Alternatively, the output of the light source 12 can be periodically modulated (for example having a sinusoidally varying amplitude) which means that the fluorescence light is also modulated. However, there is a phase lag introduced in the fluorescent emitted light because of the time dependent behaviour of the fluorescent dye or other excitable material. This manifests itself as a phase delay between the modulation of the excitation light and the modulation of the fluorescence light. The phase delay can be measured and is related to the fluorescence lifetime and hence the analyte concentration, as is known in the art.

The emitted fluorescence light is transmitted to a detector 20, again using free-space optics or a waveguide such as an optical fiber. In the embodiment shown in FIG. 1, the optical fiber 14 includes a splitter to direct some of the fluorescence light to the detector 20. An optical filter (not shown) may be provided to restrict the wavelengths of light that can reach the detector 20, for instance to block substantially all light except that at the fluorescence wavelength of interest. The detector 20 is a photodiode or other suitable light detector.

The output of the detector 20 is fed to the controller 10, which also constitutes a signal processor. In use, the lifetime and intensity of the emitted fluorescence light are inversely proportional to the concentration of the analyte at the probe tip 16 (in this embodiment the analyte is oxygen either in gaseous form or dissolved in a liquid) according to the Stern-Volmer relation. The signal processing performed in the controller 10 analyses the fluorescence light considering either or both of the intensity and lifetime (the lifetime being measured directly by intensity measurement or indirectly through phase delay measurement as explained above and as known in the art) to obtain a value quantifying the concentration of analyte at the probe tip 16. The analysis could be, for example, by direct calculation using a known mathematical relationship, or by obtaining a value from a look-up table. The measurement result is then output and can be displayed on a display (not shown) and/or can be logged in a memory (not shown) for later retrieval.

The controller 10, which incorporates the signal processor, can be implemented in dedicated electronic hardware, or in software running on a general purpose processor, such as in a personal computer, or could be a combination of the two.

FIG. 2 shows a greatly magnified view of the distal end of the optical fiber 14 and the probe tip 16 according to this preferred embodiment. On the end surface of the fiber the fluorescent material is provided in the form of nano-particles 30 in a matrix 32. The nano-particles comprise a polymer-metal complex, which in this embodiment is a PMMA-platinum (II) complex. This may also be referred to as a nano-particle dye. This exhibits a fluorescence that is quenched in the presence of oxygen. The matrix 32 is silicone. The probe tip 16 can be fabricated by dipping the end of the fiber 14 in silicone dissolved in a solvent to coat the end of the fiber, then dipping the end of the fiber in a supply of the nano-particles. The solvent then evaporates and the nano-particles are left encapsulated in the silicone matrix.

In the preferred embodiment, the nano-particles 30 are substantially spherical and have a mean diameter in the range of from 100 nm to 1000 nm. In other preferred embodiments, the maximum dimension of the nano-particles is in the range from 100 nm to 900 nm, but can be smaller. Suitable nano-particles are obtainable in powder form from “microParticles GmbH”, Berlin, Germany. Different polymer-metal complexes are also envisaged for the nano-particles, such as the metal species being Pt, Pd or Ru, or the polymer being PMMA or PEMA. The nano-particles are typically of uniform composition throughout, comprising the metal complex fluorophores embedded in the polymer and evenly distributed.

In this embodiment, the thickness of the matrix 32 is approximately 50 μm, but could be, for example in the range from 20 μm to 100 μm. Examples of suitable materials for the matrix 32 include silicone, PMMA, PEMA, and PMMA-co-styrene.

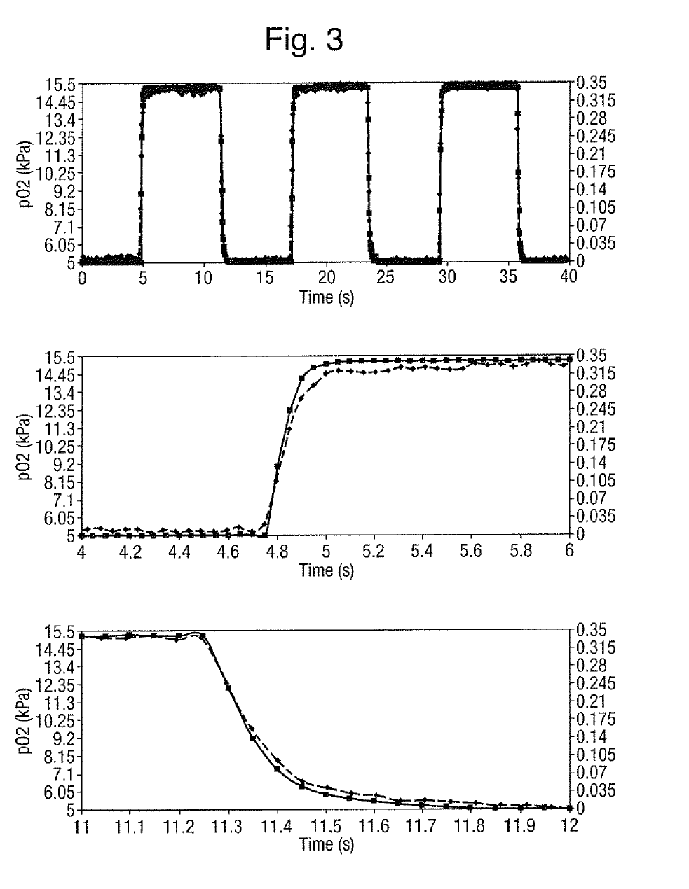

Experimental verification of the performance of the sensor is illustrated in FIG. 3. It is difficult to rapidly and controllably change the composition of a gas, such as changing the proportion of oxygen it contains. However, the sensor of this invention actually responds to the partial pressure of oxygen, rather than the relative proportion of oxygen. Therefore changing the overall gas pressure changes the oxygen partial pressure. FIG. 3 shows three graphs at different horizontal timescales for a sample cell in which the total gas pressure was cycled between two values. The black squares are data points showing the oxygen partial pressure on the left hand axis inferred from pressure measurements in which the oxygen partial pressure (pO₂) is cycled between approximately 5 and 15 kPa. The probe tip 16 of a sensor embodying the invention is located within this pressure cell. The response of the sensor is plotted on these graphs with the small grey diamonds being the data points corresponding to the right hand axis. The right hand axis is in arbitrary units and has been scaled and shifted to provide a calibration approximately corresponding with the data points plotted on the left hand axis. As can be seen, there is almost no time lag between the detection values of the sensor of the invention using fluorescence measurement and the actual change in partial pressure of oxygen in the sample cell. In particular, the response time is of the order of 200 ms or lower.

The sensor of the invention can be used in many applications, including medical, environmental and industrial monitoring.

The invention has been described with reference to various specific embodiments and examples, but it should be understood that the invention is not limited to these embodiments and examples.

1. An analyte sensor comprising:
a light source;
a probe arranged to receive light emitted from the light source;
a detector arranged to receive fluorescence light emitted from said probe in response to the light incident from the light source, and to generate an output signal; and
a signal processor arranged to determine information related to the presence of an analyte at the probe based on at least the output signal of the detector,
wherein the probe comprises nano-particles comprising fluorescent material for which the fluorescence changes in response to the presence of analyte.

2. An analyte sensor according to claim 1, wherein the maximum dimension of the nano-particles is less than 900 nm.

3. An analyte sensor according to claim 1, wherein the maximum dimension of the nano-particles is less than 100 nm.

4. An analyte sensor according to claim 1, wherein the fluorescent material comprises a polymer-metal complex.

5. An analyte sensor according to claim 4, wherein the polymer is one of or a combination of PMMA or PEMA.

6. An analyte sensor according to claim 4, wherein the metal species of each metal complex comprises any of Pt, Pd and Ru.

7. An analyte sensor according to claim 1, wherein the nano-particles are encapsulated in a matrix.

8. An analyte sensor according to claim 7, wherein the matrix comprises any of silicone, PMMA, PEMA, and PMMA-co-styrene.

9. An analyte sensor according to claim 1, wherein the nano-particles are provided in a layer.

10. An analyte sensor according to claim 9, wherein the thickness of the layer is in the range of from 10 to 100 μm.

11. An analyte sensor according to claim 1, wherein the probe comprises an optical fiber for conveying said light to and from the nano-particles which are provided on one end of the fiber.

12. An oxygen sensor comprising an analyte sensor according to claim 1, wherein the analyte is oxygen.
